(19) United States
(12) Patent Application Publication
(10) Pub. No.: US 2019/0060221 A1
(43) Pub. Date: Feb. 28, 2019

(54) TOPICAL FORMULATION OF APREMILAST

(71) Applicant: CADILA HEALTHCARE LIMITED, Ahmedabad (IN)

(72) Inventors: Amit Arvind JOHARAPURKAR, Ahmedabad (IN); Brijesh Kantilal SUTARIYA, Ahmedabad (IN); Mukul R. JAIN, Ahmedabad (IN); Sushrut Krishnaji KULKARNI, Ahmedabad (IN); Kiran R. HOTHUR, Ahmedabad (IN); Rajesh Prabhamal SIRWANI, Ahmedabad (IN)

(21) Appl. No.: 16/106,123

(22) Filed: Aug. 21, 2018

(30) Foreign Application Priority Data
Aug. 22, 2017 (IN) 201721029757

(51) Int. Cl. A61K 9/00; A61K 31/4035; A61K 47/14; A61K 45/06; A61K 9/06; A61K 47/18; A61K 47/10; A61K 47/44; A61K 9/12; A61K 47/26

(52) U.S. Cl. CPC A61K 9/0014; A61K 31/4035; A61K 47/14; A61K 45/06; A61K 47/26; A61K 47/18; A61K 47/10; A61K 47/44; A61K 9/122; A61K 9/06

(57) ABSTRACT

Present invention relates to a topical pharmaceutical composition of apremilast or its pharmaceutical acceptable salts in range of 0.01 to 25% w/w, isopropyl myristate and optionally with one or more pharmaceutically acceptable excipients and process for the preparation thereof. Further, topical formulations of this invention are therefore suitable for the treatment of psoriasis and psoriatic arthritis and they may also be used with some other medicaments.

### FIELD OF INVENTION

The present invention relates to a topical formulation comprising apremilast in the treatment of psoriasis, psoriatic arthritis, atopic dermatitis and chronic eczema. The composition of the present invention may be used alone or in combination with other systemic or topical therapies. The present invention also describes a process of producing topical pharmaceutical composition. Topical formulations may include include gels, ointments, creams, solution and foams and processes for their preparation.

### BACKGROUND OF THE INVENTION

Psoriasis is a disease where skin inflammation occurs, manifested by papules and scales, due to an abnormal homeostasis between epidermis and immune system which leads to premature maturation of keratinocytes and inflammatory cell infiltration in the dermis and abnormal hyperproliferation of the skin's epidermal layer (Griffiths et al, Lancet 2007; 370: 263-71). Abnormal epidermal hyperplasia is a major histological manifestation of psoriasis which occurs due to abnormal modulation of angiogenesis pathway VEGF/angiopoietin/Tie pathway by TNF-alpha (Kuroda et al, J Invest Dermatol 2001). In addition, the pathogenesis of psoriasis involves dendritic cells and T-cells, and cytokines like IL-6, IL-8, and IFN-gamma (Davidovici et al, J Invest Dermatol. 2010; 130(7):1785-96), apart from genetic and environmental factors. Most common clinically evident psoriasis is chronic plaque or psoriasis vulgaris. Psoriasis is associated with much comorbidity, of which psoriatic arthritis is most common (Helliwell et al, Ann Rheum Dis 2005).

Based on the pathophysiological understanding, preclinical research and clinical outcome studies, many pharmacological therapies for psoriasis have been identified and developed. They include topical treatments that involve vitamin D and its analogues, corticosteroids, calcineurin inhibitors, dithranol, and tar, phototreatments, and oral or injectable therapies that involve methotrexate, ciclosporin, retinoids, and fumarates. In the recent past, biological agents that target tumour necrosis factors α, interleukin 12, or interleukin 23, have been developed (Griffiths et al, Lancet 2007; 370: 263-71). However, the long-term safe control of psoriasis is limited due to variations in individual responsiveness, comorbidities, and side effects of the existing therapies. In addition, toxicity and patient compliance of the injectable therapies (which include biological agents) remains an issue in the long term therapeutic success.

Phosphodiesterase (PDE) 4 inhibitors are a class of low molecular weight compounds that exhibit anti-inflammatory effects (Spina 2008). Clinically, PDE4 inhibitors have been used for the treatment of various chronic inflammatory diseases, including psoriasis, psoriatic arthritis, atopic dermatitis, and inflammatory bowel disease (Schafer et al 2012). Apremilast is an oral PDE4 inhibitor that works intracellularly to regulate production of pro- and anti-inflammatory mediators implicated in the pathogenesis of psoriasis and psoriatic arthritis. Apremilast is clinically used for the treatment of adult patients with active psoriatic arthritis and for patients with moderate to severe plaque psoriasis (Young et al 2016).

Apremilast is presently available in market under the tread name of OTEZLA® by the Celgene group. OTEZLA® is marketed orally in 10 mg, 20 mg and 30 mg strength. Apremilast is generally well tolerated orally, with the most common adverse events being weight loss, diarrhea, nausea and emesis in the first year of treatment and nasopharyngitis and upper respiratory tract infection with continued treatment (Keating et al 2017). These side effects are dose-limiting and thus the therapeutic potential of apremilast may not be fully realized. To overcome this obstacle, we have utilized the topical route of administration of apremilast with potent and long-lasting local effect and minimal systemic exposure.

WO 2017168433 discloses pharmaceutical composition comprising apremilast and dimethyl sulfoxide. Another application WO 2017216738 discloses topical pharmaceutical composition comprising apremilast and solubilizers like dimethyl isosorbide.

The treatment according to the present invention consists of the topical administration of apremilast at concentrations of 0.01% w/w to 25% w/w , in the form of a gel, lotion, cream or ointment, optionally, also in combination with other active principles such as vitamins D, E and A or derivatives thereof, methotrexate or derivatives thereof, cyclosporine or derivatives thereof, palladium and/or ruthenium or derivatives thereof, anti-inflammatory agents, immunosuppressive agents, antihistamines, monoclonal antibodies and fusion proteins, cytokines and mediators, antibiotic or derivatives thereof, antifungal agents or derivatives thereof, hormones, peroxisome proliferator-activated receptor gamma activators or derivatives thereof, anti-viral agents or derivatives thereof, vegetable and/or animal extracts, phytochemicals or derivatives thereof, zinc pyrithione and/or other essential minerals, phototherapy and cell hyperproliferation modulators.

### SUMMARY OF THE INVENTION

The present invention discloses a topical formulation comprising apremilast, present at a concentration of 0.01% w/w to 25% w/w with Isopropyl myristate. Topical formulations include gel, ointment, creams, solution and foams and process for the preparation thereof. The topical formulations of this invention are therefore suitable for the treatment of psoriasis and psoriatic arthritis.

### DESCRIPTION OF FIGURES

FIG. 1:—IMQ-induced psoriasis type morphological features in mice

FIG. 2:—Topical Apremilast gel (5 mg) of the present invention, reduces severity of IMQ induced psoriasis type morphological features in mice

FIG. 3:—Topical Apremilast gel (6 mg) of the present invention reduces severity of IMQ induced psoriasis type morphological features in mice

FIG. 4:—Topical Apremilast gel (10 mg) of the present invention reduces severity of IMQ induced psoriasis type morphological features in mice

FIG. 5:—Dermis concentration of apremilast gel

### EMBODIMENTS OF THE INVENTION

The main objective of the present invention is to provide topical pharmaceutical composition comprising apremilast present at a concentration of 0.01 to 25% w/w with Isopropyl myristate and optionally with other pharmaceutically acceptable excipients

In a first embodiment is provided topical pharmaceutical composition in a gel form comprising apremilast, present at a concentration of 0.01% w/w to 25% w/w with Isopropyl myristate, optionally with other pharmaceutically excipients and process for the preparation thereof.

In a second embodiment is provided topical pharmaceutical composition in an ointment form comprising apremilast, present at a concentration of 0.01% w/w to 25% w/w with Isopropyl myristate, optionally with other pharmaceutically acceptable excipients and process for the preparation thereof.

In a third embodiment is provided topical pharmaceutical composition of in a foam form comprising apremilast, present at a concentration of 0.01% w/w to 25% w/w with Isopropyl myristate, optionally with other pharmaceutically acceptable excipients and process for the preparation thereof.

In a fourth embodiment is provided topical pharmaceutical composition in a solution form comprising apremilast, present at a concentration of 0.01% w/w to 25% w/w with Isopropyl myristate, optionally with other pharmaceutically acceptable excipients and process for the preparation thereof.

In a fifth embodiment is provided topical pharmaceutical composition of in a cream form comprising apremilast, present at a concentration of 0.01% w/w to 25% w/w with Isopropyl myristate, optionally with other pharmaceutically acceptable excipients and process for the preparation thereof.

In one of the preferred embodiment is provided a gel formulation comprising apremilast present at a concentration of 0.01% w/w to 10% w/w with Isopropyl myristate, optionally with other pharmaceutically acceptable excipients and process for the preparation thereof.

In other preferred embodiment, other pharmaceutically acceptable excipients may be selected from thickening agents, solvents, neutralization agent for polymer, vehicle, ointment base, propellant, foaming agent, solubility enhancer and surfactant and the like.

In another embodiment is provided the use of different formulations such as gel, ointment, creams, solution and foams and the like of apremilast present at a concentration of 0.01% w/w to 25% w/w according to the present invention, for the treatment of psoriasis and psoriatic arthritis.

In yet other embodiments are provided topical formulations comprising the apremilast of the present invention in combination with another medicament.

### DESCRIPTION OF THE INVENTION

The present invention relates to a topical formulation comprising apremilast or analogs and suitable pharmaceutically acceptable excipients.

The main objective of the present invention is to provide topical pharmaceutical composition comprising apremilast present at a concentration of 0.01 to 25% w/w with a suitable penetration enhancer, optionally with other pharmaceutically acceptable excipients.

### Formulation of Apremilast Gel

In a first embodiment is provided topical pharmaceutical composition in a gel form comprising apremilast, present at a concentration of 0.01% w/w to 25% w/w with Isopropyl myristate, optionally with other pharmaceutically excipients and process for the preparation thereof.

In a first embodiment is provided topical pharmaceutical composition of a gel form comprising apremilast present at a concentration of 0.01% w/w to 25% w/w with Isopropyl myristate as the penetration enhancer, optionally with other pharmaceutically excipients wherein other pharmaceutically acceptable excipients are thickening agents, neutralizing agents for polymer, vehicle, solvent, etc.

In an embodiment, other excipients such as aliphatic alcohols, monohydric/polyhydric alcohols, and mixtures thereof, can also be used as penetration enhancer.

Thickening agents used may be selected from the group of hydrophilic polymers such as carbomers, carbomer polymer, carbomer derivative (for example carbopol 974P, carbopol 934P, etc.) cellulose derivatives, anionic polymers, polyvinyl alcohol, water soluble polysaccharide may be at least one selected from the group consisting of hyaluronic acid, hyaluronate, poly-γ-glutamic acid, poly-γ-glutaminate, agar, alginic acid, alginate, carrageenan, furcellaran, pectin, arabic gum, karaya gum, tragacanth gum, ghatti gum, guar gum, locust bean gum, psyllium seed gum, gleatin, chitin, dextran, xanthan gum, chitosan, chondroitin-4-sulfate, chondroitin-6-sulfate and starch and suitable mixtures thereof.

Neutralizing agent for polymer used may be selected from the group of polyfunctional amine such as triethanolamine, poly (ethyleneimine), diisopropanolamine, triisopropanolamine, arginine, aminomethyl propanol, tetrahydroxypropyl ethylenediamine, tromethamine; alkali and alkaline earth metal hydroxides such as sodium and potassium hydroxides and lithium hydroxide, ammonium hydroxide and the like or suitable combinations thereof.

The epoxy cross-linking agent may be at least one selected from the group consisting of epichlorohydrin, 1,4-butandiol diglycidyl ether, ethylene glycol diglycidyl ether, 1,6-hexanediol diglycidyl ether, propylene glycol diglycidyl ether, polyethylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, trimethlypropane polyglycidyl ether, 1,2-(bis(2,3-epoxypropoxy)ethylene, pentaerythritol polyglycidyl ether and sorbitol polyglycidyl ether or their suitable combinations.

Vehicle is selected from purified water, suitable solvent(s) and suitable mixtures thereof.

Solvent used may be selected from the group of pharmaceutically acceptable Glycols (for example, polyethylene glycol, propylene glycol and hexylene glycol), alcohols, polysorbate 40 (a polyhydroxy organic compound), Poloxamer and the like.

General Process of Preparation:

Thickening agent is dissolved in hot water. Separately, apremilast is dissolved in suitable solvent and mixed with dissolved thickening agent. Further neutralization agent and penetration enhancer is mixed and allow to set in gel form.

### Formulation of Apremilast Ointment

In a second embodiment is provided topical pharmaceutical composition in an ointment form comprising apremilast, present at a concentration of 0.01% w/w to 25% w/w with Isopropyl myristate, optionally with other pharmaceutically acceptable excipients and process for the preparation thereof.

In a second embodiment is provided topical pharmaceutical composition of an ointment form comprising apremilast, present at a concentration of 0.01% w/w to 25% w/w with Isopropyl myristate, optionally with other pharmaceutically acceptable excipients, wherein such other pharmaceutical excipients are selected from vehicle for active, ointment base, preservative and other suitable pharmaceutically acceptable carriers.

Isopropyl myristate is used as a penetration enhancer. Other excipients such as, monohydric/polyhydric alcohols, and mixtures thereof, can also be used as penetration enhancer. Suitable vehicle is selected from the group of pharmaceutically acceptable glycols (for example, propylene glycol and hexylene glycol, diethylene glycol monoethyl ether) alcohols, polysorbate 40 (a polyhydroxy organic compound), 1,2-, diols, and mixtures thereof.

Ointment bases is selected from the group of hydrocarbon bases (for example, white petrolatum and white ointment), absorption bases (for example, hydrophilic petrolatum and lanolin), water removable bases (for example, hydrophilic ointment also known as cream), water soluble bases (for example, propylene glycol, polyethylene glycol); polyol ethers and such as white wax, stearyl alcohol, cholesterol, cetyl esters wax, and mixtures thereof.

Preservatives are selected from suitable acids such as citric acid, benzoic acid, esters, alcohols such as bronopol chlorobutanol, monothioglycerol, phenols such as cresol, Butylated hydroxylanisol, Butylated hydroxyltoluene, and quaternary ammonium compounds.

General Process of Preparation:

Ointment base is heated and stirred till clear solution is obtained. Separately, apremilast is dissolved in a suitable solvent and added to melted ointment base. Lastly a preservative and isopropyl myristate is added to the solution and allowed to cool till the dispersion to form ointment.

### Formulation of Apremilast Quick Breaking Foam

In a third embodiment is provided topical pharmaceutical composition in a foam form comprising apremilast, present at a concentration of 0.01% w/w to 25% w/w with a suitable penetration enhancer, optionally with other pharmaceutically acceptable excipients and processes for the preparation thereof.

In a third embodiment is provided a topical pharmaceutical composition in a foam form comprising apremilast, present at a concentration of 0.01% w/w to 25% w/w with a suitable penetration enhancer, optionally with other pharmaceutically acceptable excipients, wherein other pharmaceutical excipients are selected from a propellant, humectant, foaming agent, solubility enhancer for foaming agents, pH modifiers, buffers, hydrotropic agents, solvents and other pharmaceutically acceptable carriers.

According to the invention, isopropyl myristate is used as the penetration enhancer. Other excipients such as aliphatic alcohols, monohydric/polyhydric alcohols, and mixtures thereof, can also be used as penetration enhancer. Propellant may be selected from the group consisting of hydrocarbon, a chlorofluorocarbon dimethyl ether, hydroflourocarbon and mixture thereof.

Foaming agent may be selected from the group of block copolymer, fatty acid ethoxylates, fatty alcohol ethoxylates, polysorbates and glycerol ester ethoxylates and their suitable mixtures.

Solubility enhancer for foaming agents Non Volatile Liquids: Poly Ethylene Glycol 200, Poly Ethylene Glycol 300, Poly Ethylene Glycol 400, Glycerine, Propylene Glycol, fixed oils and their suitable mixtures.

Hydrotropic agents may be selected from suitable aromatic anionics or cationics. Aromatic anionics used may be selected from Sodium benzoate, Sodium salicylate, Sodium benzene sulphonate, Sodium benzene disulphonate, Sodium cinnamate or their suitable mixtures.

Aromatic cationics used may be selected from para amino benzoic acid hydrochloride, Procaine hydrochloride, Caffeine. Aliphatics and linear anionics Sodium alkanoate or their suitable mixtures.

Solvents used may be a polar hydrophilic solvent, which may be selected from the following groups of compounds: (1) polyols (organic solvents that contain at least two hydroxy groups in their molecular structure); and (2) polyethylene glycols (PEGs). The polyols can be selected from the group of di-alcohols, such as propylene glycol, butanediol and diethylene glycol, and tri-alcohols, such as glycerin. The PEGs can be primarily low-molecular weight liquid PEGs, such as PEG 200, PEG 400, PEG 600 and PEG 1000; however, mixes of the liquid PEGs with higher molecular weight such as PEG 4000, PEG 6000, and PEG 8000 .

Secondary polar solvents may be selected from dimethyl isosorbide, ethoxydiglycol, and alpha hydroxy acids, such as lactic acid and glycolic acid.

Highly purified diethylene glycol monoethyl ether (e.g. Transcutol® P) can also be selected as solvent in foam.

pH modifiers can be selected from inorganic salts e.g. calcium chloride; barium chloride; or from acids, especially organic acids, salts, buffering substances, e.g. citric acid, acetic acid, salicylic acid, lactic acid and other alpha hydroxy acids as well as inorganic acids e.g. HCl, NaOH, KOH and other bases. These regulators are used in amounts sufficient to provide the desired pH value of the composition

Buffer may be selected from the group containing citrate/citric acid, acetate/acetic acid, phosphate/phosphoric acid, formate/formic acid, propionate/propionic acid, lactate/lactic acid, carbonate/carbonic acid and ammonium/ammonia buffer and phosphate buffers.

Aqueous quick breaking foam agent may be selected from C₁-C₆ alcohol.

General Process of Preparation:

Quick breaking foam is prepared by dissolving Apremilast in solvent/ humectant and solubility enhancer was added by dissolving in water. Further buffer/ pH modifier was added to this with continuous stirring.

In separate vessel foaming agent is melted and mixed with above solution. At last, propellant and Isopropyl myristate is added upon cooling.

### Formulation of Apremilast Solution

In a fourth embodiment is provided topical pharmaceutical composition in a solution form comprising apremilast, present at a concentration of 0.01% w/w to 25% w/w with Isopropyl myristate, optionally with other pharmaceutically acceptable excipients and process for the preparation thereof.

In a fourth embodiment is provided topical pharmaceutical composition in a solution form comprising apremilast, present at a concentration of 0.01% w/w to 25% w/w with Isopropyl myristate, optionally with other pharmaceutically acceptable excipients; wherein other pharmaceutically acceptable excipients are selected from suitable preservatives, solvent for active, a suitable vehicle and other pharmaceutically acceptable carriers.

Isopropyl myristate is used as the penetration enhancer. Other excipients such as aliphatic alcohols, monohydric/polyhydric alcohols or mixtures thereof, can also be used as penetration enhancer. Preservatives are selected from acids, esters, alcohols, phenols, mercurial compounds and quaternary ammonium compounds.

Solvents for active are selected from 1,2-, diols, polyether compounds and mixtures thereof.

General Process of Preparation:

Solution are prepared by dissolving the apremilast in a solvent as described above, mixing until dissolved, then making up to the desired volume by adding sufficient solvent. Other excipients may be added to the solvent that help stabilize or solubilize the active ingredient, which are well known to a person skilled in the art.

In one of the preferred embodiment is provided a gel formulation comprising apremilast present at a concentration of 0.01% w/w to 10% w/w with isopropyl myristate and optionally with other pharmaceutically acceptable excipients and process for the preparation thereof.

In specifically preferred embodiment is provided a gel formulation comprising apremilast present at 2.5% w/w, 3.0% w/w and 5.0% w/w concentration with Isopropyl myristate and optionally with other pharmaceutically acceptable excipients and process for the preparation thereof.

In another preferred embodiment, other pharmaceutically acceptable excipients may be selected from thickening agents, solvents, neutralization agent for polymer, vehicle, ointment base, propellant, foaming agent, solubility enhancer and surfactant, etc.

Isopropyl myristate is used as a Penetration enhancer.

Thickening agents used may be selected from the group of hydrophilic polymers such as carbomers, carbomer polymer, carbomer derivative (for example carbopol 974P, carbopol 934P, etc.) cellulose derivatives, anionic polymers, polyvinyl alcohol, water soluble polysaccharide may be at least one selected from the group consisting of hyaluronic acid, hyaluronate, poly-γ-glutamic acid, poly-γ-glutaminate, agar, alginic acid, alginate, carrageenan, furcellaran, pectin, arabic gum, karaya gum, tragacanth gum, ghatti gum, guar gum, locust bean gum, psyllium seed gum, gleatin, chitin, dextran, xanthan gum, chitosan, chondroitin-4-sulfate, chondroitin-6-sulfate and starch and mixtures thereof, preferably carbomer derivatives.

Neutralizing agent for polymer used may be selected from the group of polyfunctional amine such as triethanolamine, poly (ethyleneimine), diisopropanolamine, triisopropanolamine, arginine, aminomethyl propanol, tetrahydroxypropyl ethylenediamine, tromethamine; alkali and alkaline earth metal hydroxides such as sodium and potassium hydroxides and lithium hydroxide, ammonium hydroxide and the like, preferably triethanolamine.

Vehicle is selected from purified water, solvent and mixture thereof.

Solvent used may be selected from the group of pharmaceutically acceptable Glycols (for example, polyethylene glycol, propylene glycol and hexylene glycol), alcohols, polysorbate 40 (a polyhydroxy organic compound), Poloxamer, preferably polyethylene glycol and propylene glycol.

General Process of Preparation:

Gel is prepared by dissolving (i) 1.5% w/w thickening agent in hot water at 70° C. and (ii) 0.01-10% w/w apremilast in solvent (or mixture of solvent) and then mixing (i) and (ii) to make solution (iii). To the solution of step (iii) 0.75% w/w neutralizing agent for polymer and 4.0% w/w penetration enhancer is added and allowed it to set in gel form.

The formulations of the present invention such as the gel, ointment, creams, solution and foams of apremilast can be used for the treatment of psoriasis and psoriatic arthritis.

In another further embodiment the topical formulation of the invention described above may optionally be combined with another medicament. In an embodiment, the pharmaceutical composition as described above can be combined with vitamins D, E and A or derivatives thereof, methotrexate or derivatives thereof, cyclosporin or derivatives thereof, palladium and/or ruthenium or derivatives thereof, anti-inflammatory and immunosuppressive agents inclusive of (but not limited to JAK inhibitors, IKK-TBK inhibitors, PDE3 AND PDE4 inhibitors), antihistamines, monoclonal antibodies and fusion proteins, cytokines and other hormone mediators, antibiotic or derivatives thereof, antifungal agents or derivatives thereof, hormones, peroxisome proliferator-activated receptor gamma activators or derivatives thereof, NSAIDs or analogs thereof, anti-viral agents or derivatives thereof, vegetable and/or animal extracts, phytochemicals or derivatives thereof, zinc Pyrithione and/or other essential minerals, phototherapy and cell hyperproliferation modulators.

### EXAMPLES

The invention is explained in greater detail by the examples given below, which are provided by way of illustration only and therefore should not be construed to limit the scope of the invention.

### Example 1

Process for the Preparation of Apremilast Gel

1. Heat Purified water upto 70° C. and dissolve Carbopol 974P in it under stirring.

2. Heat Polyethlyene glycol/propylene glycol upto 50° C. and dissolve Apremilast in it.

3. Add solution of step 2 to step 1 under stirring and allow to stir well.

4. Add Triethanolamine and Isopropyl myristate to step 3 under stirring and allow to stir till gel formation.

5. Allow to cool the gel and fill in Aluminum tubes.

| | | | Formulation | | |
|---|---|---|---|---|---|
| | | | 1 | 2 | 3 |
| Sr. No. | Ingredient Name | Function | Amount (% w/w) | | |
| 1 | Apremilast | Active Pharmaceutical Ingredients | 2.5 | 3.0 | 5.0 |
| 2 | Carbopol 974P | Thickening agent | 1.5 | 1.5 | 1.5 |
| 3 | PEG 400 | Solvent for Active | 20.0 | 20.0 | 20.0 |
| 4 | Propylene glycol | Solvent for Active | 10.0 | 10.0 | 10.0 |
| 5 | Triethanolamine | Neutralizing agent for polymer | 0.75 | 0.75 | 0.75 |
| 6 | Isopropyl Myristate | Penetration enhancer | 4.0 | 4.0 | 4.0 |
| 7 | Purified Water | Vehicle/Solvent | q.s. | q.s | q.s |

### Example 2

Process for the Preparation of Apremilast Ointment:

(i) Heat White petrolatum upto 60° C. to form clear solution.

(ii) Stir solution of step 1.

(iii) Dissolve/disperse apremilast in transcutol/mineral oil and add to step 2 under stirring.

(iv) Add butylated hydroxy anisole and isopropyl myristate to step 3 under stirring.

(v) Allow to cool the dispersion to form ointment and then fill in Aluminum tubes.

| | | | Formulation | | | | |
|---|---|---|---|---|---|---|---|
| | | | 1 | 2 | 3 | 4 | 5 |
| Sr. No | Ingredient name | Function | Amount (% w/w) | | | | |
| 1 | Apremilast | Active | 1.00 | 3.00 | 5.00 | 1.00 | 1.00 |
| 2 | Mineral oil | Vehicle for Active | 30.00 | 30.00 | 30.00 | 30.00 | 30.00 |
| 3 | Transcutol | Vehicle for Active | 5.00 | 5.00 | 5.00 | 5.00 | 5.00 |
| 4 | White petrolatum | Ointment base | 62.00 | 58.00 | 56.00 | 63.00 | 63.00 |
| 5 | Isopropyl Myristate | Penetration enhancer | 2.0 | 4.0 | 4.0 | — | — |
| 6 | Butylated Hydroxy Anisole/ | Preservative | — | — | — | 1.00 | 1.00 |
| | Butylated Hydroxy Toluene | | | | | | |

### Example 3

Process of Apremilast Quick Breaking Foam

(i) Heat Propylene glycol up to 50° C. and dissolve Apremilast in it.

(ii) Dissolve Polysorbate 80 in purified water under stirring.

(iii) Step 2 added step 1 under continuous stirring.

(iv) Added potassium hydroxide solution in step 3 under continuous stirring.

(v) In a separate vessel, melt together cetyl alcohol & steryl alcohol and heat to 75° C.

(vi) Add the later mixture to the aqueous phase with continuous stirring.

(vii) When cooled to 40° C. mix the dehydrated alcohol, propane, butane, isopropyl myristate, and added slowly to the emulsion with stirring and discontinue stirring when temperature reached to 30°.

| | | | Formula- | Recommended |
|---|---|---|---|---|
| Sr. | | | tion 1 | conc. ranges |
| No | Ingredient name | Function | Amount (% w/w) | |
| 1 | Apremilast | Active | 1.00 | 0.1-5 |
| 2 | Dehydrated | Solvent for API | 58.21 | 40-60 |
| | alcohol | and foaming agent | | |
| 3 | Cetyl alcohol | Foaming agent | 1.20 | 0.5-2.0 |
| 4 | Stearyl alcohol | Foaming agent | 0.50 | 0.25-4 |
| 5 | Polysorbate 80 | Solubility enhancer | 0.50 | 0.25-4 |
| | | for foaming agents | | |
| 6 | Propylene | Humectant | 2.50 | 1.0-4.0 |
| | glycol | | | |
| 7 | Isopropyl | Penetration | 2.00 | 1.0-6.0 |
| | Myristate | enhancer | | |
| 8 | Purified water | Solvent | q.s. | q.s. |
| 9 | Potassium | pH modifier | 0.10 | 0.05-0.2 |
| | hydroxide, 10% | | | |
| | w/w solution | | | |
| 10 | Propane | propellant | 5.00 | 3.0-7.0 |
| 11 | Butane | propellant | 5.00 | 3.0-7.0 |

### Example 4

Process for the Preparation of Apremilast Solution

1. Heat Propylene glycol upto 50° C. and dissolve Apremilast in it.

2. Dissolve butylated hydroxy anisole in polyethylene glycol under continuous stirring.

3. Step 2 added step 1 under continuous stirring.

4. In a separate vessel added isopropyl myristate in purified water under stirring.

Solution of step 4 was added to solution of step 3 under continuous stirring

| | | | Formula- | Recommended |
|---|---|---|---|---|
| Sr. | | | tion 1 | conc. ranges |
| No | Ingredient name | Function | Amount (% w/w) | |
| 1 | Apremilast | Active | 1.00 | 0.1-5 |
| 2 | Polyethylene | Solvent for Active | 5.00 | 2.0-7.0 |
| | glycol | | | |
| 3 | Propylene glycol | Solvent for Active | 5.00 | 2.0-7.0 |
| 4 | Isopropyl | Penetration | 2.0 | 1.0-6.0 |
| | Myristate | enhancer | | |
| 5 | Butylated | Preservative | 0.50 | 0.1-1.0 |
| | hydroxy anisole | | | |
| 6 | Purified water | Vehicle | q.s. | q.s. |

### Example 5

Biological Studies

Animal:

Six to eight week old male C57BL6/J mice were obtained from the Animal Research Facility of Zydus Research Centre, Ahmedabad, India. They were housed in ventilated cages, in an air conditioned room with relative humidity 55±10%, subjected to 12 h dark/light cycles and given free access to food and water. The procedures for animal use and experimentation were reviewed and approved by Institutional Animal Ethics Committee (as per CPCSEA, Committee for the Purpose of Control and Supervision of Experiments on Animals, Government of India) of Zydus Research Centre, which is an AAALAC accredited facility.

Protocol:
a) Mice were administered a daily topical dose of commercially available Imiquimod (IMQ) cream (5%, Imiquad; Glenmark Pharmacetical Ltd) on the shaved back for 5 consecutive days, translating in a daily dose of 3.125 mg of the active compound to achieve IMQ-induced psoriasis.
b) All animals were assessed for the severity of the psoriasis-like skin condition, using 3 elements (erythema, scaling and thickness) of the Psoriasis Area Severity Index (PAST), to assign a score of 0-4:
   0—none
   1—mild
   2—moderate
   3—severe
   4—very severe
c) The cumulative score (erythema plus scaling plus thickness) served to indicate the severity of inflammation (scale 0-12).
d) Mice with induced psoriasis type morphological features were treated twice a daily topical formulation of 2.5%, 3% and 5% apremilast gel (100 mg on the back, i.e. 5 mg, 6 mg and 10 mg of apremilast, respectively) for 5 consecutive days.

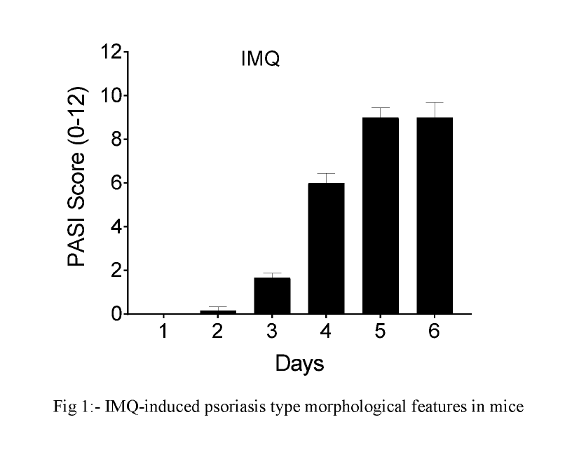

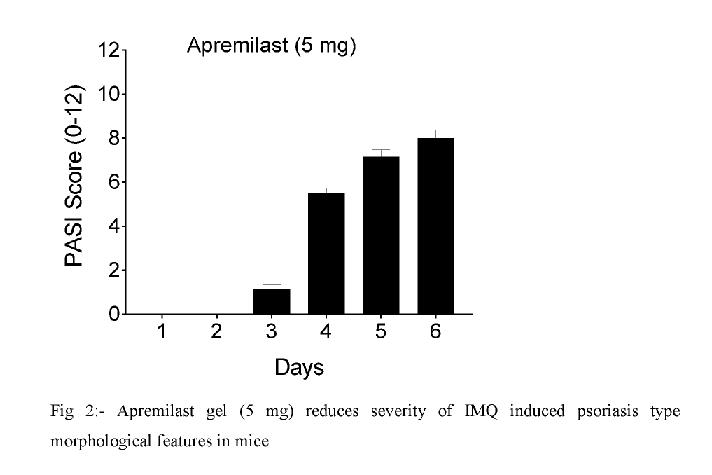

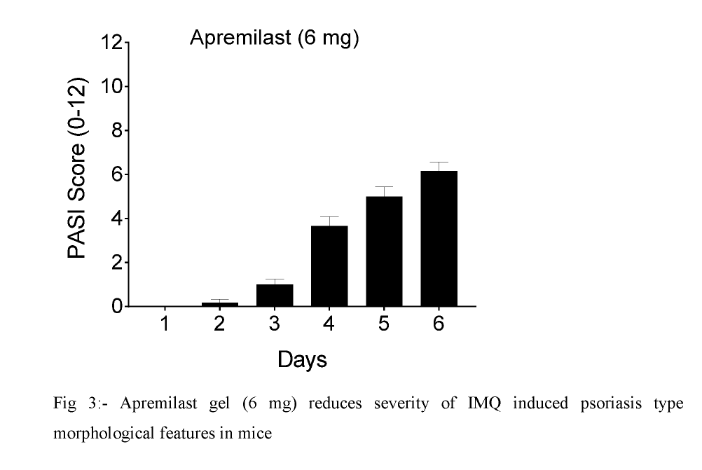

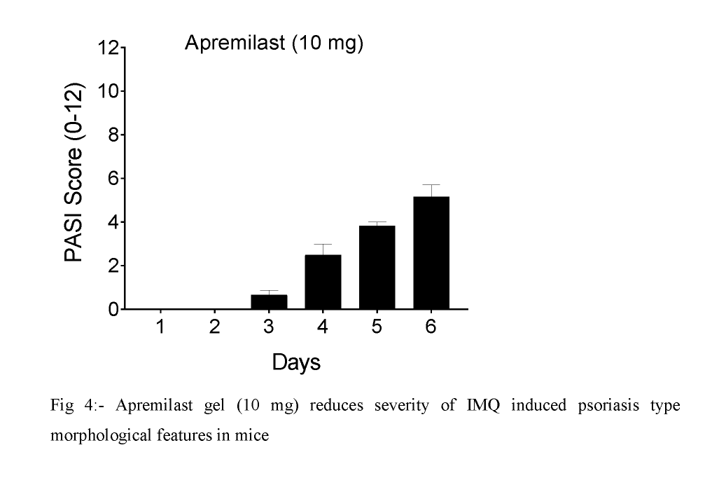

Observation:
a) As shown in FIG. 1, two to three days after the start of IMQ application, the back skin of the mice started to display signs of erythema, scaling, and thickening, which continually increased in severity up to the end of the experiment.
b) Formulations of 5 mg (FIG. 2), 6 mg (FIGS. 3) and 10 mg (FIG. 4) concentrations have reduced the signs of erythema, scaling, and thickening induced by IMQ cream.

Conclusion:
a) All the gel formulation of apremilast decreased the IMQ induced psoriasis type morphological features

Gel formulations of 6 mg and 10 mg concentration have shown superior results

### Example 6

Pharmacokinetics of apremilast in male C57BL6/J mice

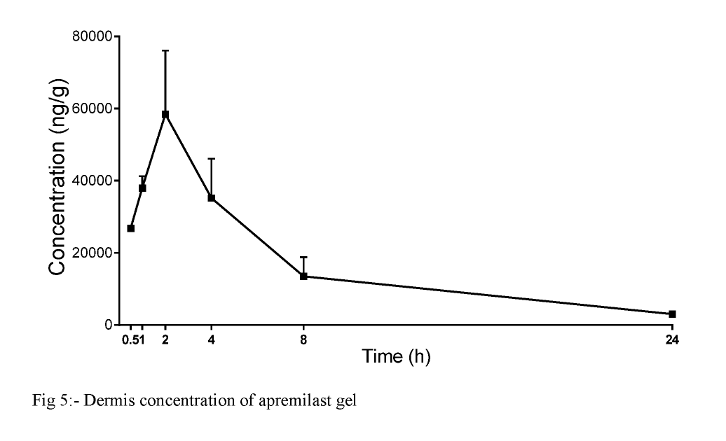

Pharmacokinetics of apremilast was studied in male C57BL6/J mice by topical route. Two day before treatment, mice hair was removed from the dorsal regions and test area (1.5 cm2) was marked. Total eighteen mice were randomly divided in six groups (n=3). The formulation was applied to the test area at an amount of 1 mg/cm2 and dermis was collected at each time points i.e. 0.5, 1, 2, 4, 8 and 24 hours post administration. FIG. 5 represents the dermis concentration of apremilast quantified by LC/MS-MS methods and PK parameters are listed in Table 1

TABLE 1
- PK parameters of apremilast

| Parameters | Topical apremilast gel |
|---|---|
| Tmax (h) | 2 |
| Cmax (ng/g) | 58452.79 |
| AUC0-last | 408596.31 |

1. Topical pharmaceutical composition of apremilast comprising apremilast and its pharmaceutical acceptable salts in range of 0.01 to 25% w/w, isopropyl myristate and optionally with other pharmaceutically acceptable excipients.

2. Topical pharmaceutical composition of apremilast as claimed in claim 1 which is present as a gel, cream, solution or quick breaking foam.

3. Topical pharmaceutical composition of apremilast claimed in claim 1 which is in gel form wherein other pharmaceutically acceptable excipients are selected from suitable, thickening agents, neutralizing agents for polymer, vehicle and solvent.

4. Topical pharmaceutical composition of apremilast as claimed in claim 3, wherein the thickening agent is selected from the group of hydrophilic polymers carbomers, carbomer polymer, carbomer derivative, cellulose derivatives, anionic polymers, polyvinyl alcohol, preferably carbomer polymer carbopol 974P.

5. Topical pharmaceutical composition of apremilast as claimed in claim 3, wherein neutralization agent for polymer is selected form triethanolamine, poly (ethyleneimine), diisopropanolamine, triisopropanolamine, arginine, aminomethyl propanol, tetrahydroxypropyl ethylenediamine, tromethamine, sodium and potassium hydroxides and lithium hydroxide, ammonium hydroxide, preferably triethanolamine.

6. Topical pharmaceutical composition of apremilast as claimed in claim 3, wherein solvent or mixture of solvent is selected from acceptable glycols derivatives selected from polyethylene glycol, propylene glycol, hexylene glycol, alcohols, polysorbate 40, poloxamer, diethylene glycol monoethyl ether, mineral oil, dehydrated alcohols selected from polyethylene glycol, propylene glycol or their suitable mixtures.

7. Topical pharmaceutical composition of apremilast claimed in claim 1 which is in ointment form wherein other pharmaceutically acceptable excipients are selected from ointment base, preservatives and vehicle for active.

8. The topical pharmaceutical composition of apremilast as claimed in claim 7, wherein ointment base is selected from white petrolatum hydrophilic petrolatum, lanolin, propylene glycol, polyethylene glycol, polyol ethers , white wax, stearyl alcohol, cholesterol, cetyl esters wax, and their suitable mixtures.

9. Topical pharmaceutical composition of apremilast as claimed in claim 7 wherein preservative is selected from acids, esters, alcohols, phenols selected from butylated hydroxy anisol, butylated hydroxy toluene, and quaternary ammonium compounds.

10. Topical pharmaceutical composition of apremilast claimed in claim 7, wherein vehicle for active is selected from group of pharmaceutically acceptable glycols selected from propylene glycol, hexylene glycol, diethylene glycol monoethyl ether, alcohols, polysorbate 40,1,2-, diols, and suitable mixtures thereof.

11. Topical pharmaceutical composition of apremilast claimed in claim 1 which is in quick breaking foam, wherein other pharmaceutically excipients are selected from propellant, foaming agents, solubility enhancer for foaming agents, solvents/humectant, pH modifier and buffer.

12. Topical pharmaceutical composition of apremilast claimed in claim 11 wherein propellant is selected from the group comprising of hydrocarbons selected from propane and butane, a chlorofluorocarbon dimethyl ether, hydroflourocarbon and suitable mixtures thereof.

13. Topical pharmaceutical composition of apremilast claimed in claim 11, wherein foaming agent is selected from of block copolymer, fatty acid ethoxylates, fatty alcohol ethoxylates selected from cetyl alcohol and steryl alcohol, polysorbates and glycerol ester ethoxylates.

14. Topical pharmaceutical composition of apremilast claimed in claim 11, wherein solubility enhancer for foaming agent is selected from Peg 200, Peg 300, Peg 400, polysorbate 80, glycerine, propylene glycol, sodium benzoate, sodium salicylate, sodium benzene sulphonate, sodium benzene disulphonate, sodium cinnamate or their suitable mixtures.

15. Topical pharmaceutical composition of apremilast claimed in claim 11, wherein solvent/humectant is selected from polyethylene glycols selected from PEG 200, PEG 400, PEG 600 and PEG 1000, propylene glycol, butane diol, diethylene glycol, purified water and their suitable mixtures.

16. Topical pharmaceutical composition of apremilast claimed in claim 11, wherein pH modifier is selected from calcium chloride, barium chloride, citric acid, acetic acid, salicylic acid, lactic acid, sodium hydroxide, potassium hydroxide and phosphate buffer

17. Topical pharmaceutical composition of apremilast claimed in claim 1 which is in solution form, wherein other pharmaceutically acceptable excipients are selected from solvent for active and preservatives.

18. Topical pharmaceutical composition of apremilast claimed in claim 17 wherein solvents for active are selected from 1,2-, diols, polyether compounds and mixtures thereof.

19. Topical pharmaceutical composition of apremilast claimed in claim 17 wherein preservative is selected from acids, esters, alcohols, phenols selected from butylated hydroxy anisol, butylated hydroxy toluene, mercurial compounds and quaternary ammonium compounds or their suitable mixtures.

20. Topical pharmaceutical composition as claimed in claim 1 for the treatment for psoriasis or psoriatic arthritis.

21. Topical pharmaceutical composition as claimed in claim 1 in combination with a second medicament.

22. Topical pharmaceutical composition as claimed in claim 1 in combination with a second medicament wherein second medicament is selected from vitamins D, E and A or its derivatives, methotrexate or its derivatives, cyclosporin or its derivatives, anti-inflammatory and immunosuppressive agents, antihistamines, monoclonal antibodies and fusion proteins, cytokines and other hormone mediators, antibiotic or its derivatives, antifungal agents or its derivatives, hormones, peroxisome proliferator-activated receptor gamma activators or derivatives thereof, NSAIDs or analogs thereof, anti-viral agents or its derivatives, zinc pyrithione and/or other essential minerals, phototherapy and cell hyperproliferation modulators.

23. Topical pharmaceutical composition of apremilast in gel form comprising apremilast and its pharmaceutical acceptable salts in range of 0.01 to 10% w/w, with isopropyl myristate as a penetration enhancer, carbopol 974P as a thickening agent (gelling agent), triethanolamine as a neutralizing agent for polymer, polyethylene glycol and propylene glycol as a solvent.
